(19) United States
(12) Patent Application Publication
(10) Pub. No.: US 2018/0144923 A1
(43) Pub. Date: May 24, 2018

(54) METHOD FOR CHARACTERISING IONS

(71) Applicants: UNIVERSITE D'AIX MARSEILLE, Marseille Cedex 07 (FR); CENTRE NATIONAL DE LA RECHERCHE SCIENTIFIQUE (CNRS), Paris Cedex 16 (FR)

(72) Inventors: Géraldine FERAUD, Vence (FR); Christophe JOUVET, Marseille (FR); Claude DEDONDER, Marseille (FR)

(21) Appl. No.: 15/572,063

(22) PCT Filed: May 4, 2016
(86) PCT No.: PCT/EP2016/060057

§ 371 Date: Nov. 6, 2017

(30) Foreign Application Priority Data
May 7, 2015 (FR) FR1554093

(51) Int. Cl. H01J 49/42; H01J 49/00; H01J 49/04; G01N 33/68

(52) U.S. Cl. CPC H01J 49/427; H01J 49/0059; G01N 33/6812; G01N 33/6848; H01J 49/0468

(57) ABSTRACT

A method for characterising ions includes trapping a first-generation ions in an ion trap; cooling the plurality of first-generation ions; photo-fragmenting the plurality of cooled first generation ions to obtain a plurality of second-generation ions, the second-generation ions being different to the first-generation ions, the plurality of second-generation ions being at least of one first type; selecting the first type of second-generation ions in the ion trap by ejecting, out of the trap, any residual first-generation ion and any second-generation ion of a type different from the first type; cooling the second-generation ions of the first type selected and trapped in the trap; photo-fragmenting the cooled second-generation ions of the first type to obtain a plurality of third-generation ions, the plurality of third-generation ions being different from the plurality of second-generation ions, the plurality of third-generation ions being at least of one first type; detecting the plurality of last-generation ions.

### TECHNICAL FIELD OF THE INVENTION

The technical field of the invention is that of ion characterisation.

A first aspect of the present invention relates to a method for characterising ions; a second aspect of the present invention relates to a device for characterising ions for the implementation of a method of characterising ions according to the first aspect of the invention.

The present invention notably finds applications in the fields of analytical chemistry, pharmacology and the environment.

### TECHNOLOGICAL BACKGROUND OF THE INVENTION

The field of mass spectrometry is currently in full expansion. Numerous industrial concerns propose instruments, notably used in the fields of analytical chemistry, pharmacology and the environment, in order to characterise systems. However, these instruments uniquely make it possible to characterise the mass/charge ratio, noted m/z, of the systems detected, and not, notably, the different isomers or tautomers of the systems detected.

Tandem mass spectrometry, noted MS/MS or MS², is a mass spectrometry technique for the identification and the characterisation of ions. The MS/MS technique consists in:
isolating in a mass spectrometer first-generation ions, having a specific mass/charge ratio;
activating the parent ions, for example by a Collision Induced Dissociation (CID) technique;
isolating a specific fragment derived from the activation of the first-generation ions;
activating said specific fragment, for example by the CID technique, in order to obtain third-generation fragments.

The study of third-generation ions makes it possible to obtain information on second-generation ions, and thus makes it possible to obtain indirectly information on first-generation ions. The study of third-generation ions may for example be carried out by means of electron spectroscopy.

Optical excitation, or photo-fragmentation, is another ion fragmentation technique that may be used. The following are typically used:
either a photo-fragmentation technique with IR photons for all types of ions,
or a visible or UV photo-fragmentation technique for ions having a chromophore.

Electron spectroscopy of an ion makes it possible to obtain a spectrum giving information on the overall structure of said ion, but it remains difficult to determine the tautomeric form of the ion, notably on account of a phenomenon of spectral congestion when the ion is hot. The phenomenon of spectral congestion reflects the fact that the spectrum obtained, which contains characteristic fine rays when the ion is cold, becomes a wide and non-characteristic band when the ion is hot. It should be noted that an ion at room temperature is typically a hot ion.

The inventors are the authors of the article “Communication: Identification of daughter ions through their electronic spectroscopy at low temperature”, The Journal of Chemical Physics (2014). This article proposes a method for characterising the isomeric structure of an ionic photo-product, according to which:
first-generation ions are trapped in an ion trap;
the trapped first-generation ions are cooled;
the cooled first-generation ions are photo-fragmented: second-generation ions are obtained;
the second-generation ions are cooled;
the cooled second-generation ions are photo-fragmented: third-generation ions are obtained;
third-generation ions are detected.

In the article, the characterisation method is applied to the specific case of protonated tyrosine TyrH+ and makes it possible to characterise structurally the different photo-products of TyrH+. The method having made it possible to characterise fragments derived from a protonated amino acid, it is envisaged in the conclusion of the article to test the method for characterising fragments derived from a protonated peptide, in a more complete manner than the characterisation obtained by mass spectrometry.

However, experiments carried out in order to apply said method to ions other than protonated tyrosine TyrH+ are not conclusive.

### SUMMARY OF THE INVENTION

The invention offers a solution to the aforementioned problems, by proposing a method of characterising ions, and in particular a method for characterising the isomeric structure of ions, the method being applicable to all types of ions.

One aspect of the invention thus relates to a method for characterising ions comprising:
a step according to which a plurality of first-generation ions is trapped in an ion trap;
a step according to which the plurality of first-generation ions trapped in the ion trap is cooled;
a step according to which the plurality of cooled first-generation ions is photo-fragmented to obtain a plurality of second-generation ions, the plurality of second-generation ions being different to the plurality of first-generation ions, the plurality of second-generation ions being at least of one first type;
a step i) according to which the first type of second-generation ions is selected in the ion trap by ejecting, out of the ion trap, any residual first-generation ion and any second-generation ion of a type different to the first type;
a step ii) according to which the second-generation ions of the first type selected and trapped in the ion trap are cooled;
a step iii) according to which the cooled second-generation ions of the first type are photo-fragmented to obtain a plurality of third-generation ions, the plurality of third-generation ions being different to the plurality of second-generation ions, the plurality of third-generation ions being at least of one first type;
a step according to which the plurality of last-generation ions is detected.

The first photo-fragmentation of the plurality of first-generation ions makes it possible to obtain the plurality of second-generation ions. The second-generation ions are photo-fragments of the first-generation ions, thus the second-generation ions are different to the first-generation ions. The second-generation ions may all be of a same type, or the second-generation ions may be of different types. “Ions of different types” is taken to mean ions that can be selected independently of each other. Moreover, first-generation ions may subsist at the end of the first photo-fragmentation. Step i) makes it possible to isolate a first type of second-generation ions in the ion trap. Thus, it is guaranteed that the second photo-fragmentation is carried out uniquely on this first type of second-generation ions. The third-generation ions are thus photo-fragments of this first type of second-generation ions. Isolating the first type of second-generation ions in the ion trap before carrying out the second photo-fragmentation enables a reliable characterisation of the first type of second-generation ions, by using the third-generation ions obtained. Indeed, the spectroscopic signature of the first type of second-generation ions is obtained by the detection of its photo-fragments, that is to say by the detection of third-generation ions. Yet ions identical to the third-generation ions may also be obtained, notably, by photo-fragmentation of residual first-generation ions or by photo-fragmentation of second-generation ions of a type different to the first type. It is also possible that ions of different generations absorb in the same spectral domain. For example, a first-generation ion and a second-generation ion may both absorb in the UV. In such cases, the reliable characterisation of the first type of second-generation ions is made impossible because the spectroscopic signature of the first type of second-generation ions is mixed with the spectroscopic signature of other ions such as first-generation ions and/or second-generation ions of a type different to the first type. Thus, the method described in the publication cited previously functions when it is applied to the specific case of protonated tyrosine TyrH+ because protonated tyrosine TyrH+ absorbs in the UV whereas second-generation ions, that is to say the photo-fragments of protonated tyrosine TyrH+, absorb in the visible.

More generally, isolating a unique type of Nth-generation ions before carrying out an Nth photo-fragmentation enables a reliable characterisation of the type of Nth-generation ions.

Apart from the characteristics that have just been mentioned in the preceding paragraph, the characterisation method according to one aspect of the invention may have one or more additional characteristics among the following, considered individually or according to all technically possible combinations thereof:
The method comprises a step according to which the plurality of first-generation ions is selected by a mass spectrometry technique.
The step according to which the plurality of last-generation ions is detected is carried out by a mass spectrometry technique.
Steps i), ii) and iii) form a sequence that is carried out N times, N being a natural integer greater than or equal to 2, the number of each generation being incremented by 1 each time said sequence is carried out.

Another aspect of the invention relates to a device for characterising ions for the implementation of the method for characterising ions according to one aspect of the invention, the device being characterised in that it comprises:
an ion trap for trapping a plurality of ions;
a module for cooling the plurality of ions, comprising a buffer gas source and a cryostat;
a photo-fragmentation laser;
a mass spectrometer for selecting a type of ions of interest;
a detector for detecting a plurality of photo-fragments.

The invention and its different applications will be better understood on reading the description that follows and by examining the figures that accompany it.

### BRIEF DESCRIPTION OF THE FIGURES

The figures are presented for indicative purposes and in no way limit the invention.

FIG. 1a shows a diagram of the steps of a method for characterising ions according to a first embodiment of the invention.

FIG. 1a′ shows a diagram of the complementary steps of the method for characterising ions according to an alternative of the first embodiment of the invention.

FIG. 1b shows a diagram of the steps of a method for characterising ions according to a second embodiment of the invention.

FIG. 2a shows schematically a first phase of use of a device for characterising ions for the implementation of a method for characterising ions according to the first or the second embodiment of the invention.

FIG. 2b shows schematically a second phase of use of a device for characterising ions for the implementation of a method for characterising ions according to the first or the second embodiment of the invention.

FIG. 2c shows schematically a third phase of use of a device for characterising ions for the implementation of a method for characterising ions according to the first or the second embodiment of the invention.

FIG. 2d shows schematically a fourth phase of use of a device for characterising ions for the implementation of a method for characterising ions according to the first embodiment of the invention.

### DETAILED DESCRIPTION OF AT LEAST ONE EMBODIMENT OF THE INVENTION

Unless stated otherwise, a same element appearing in the different figures has a single reference.

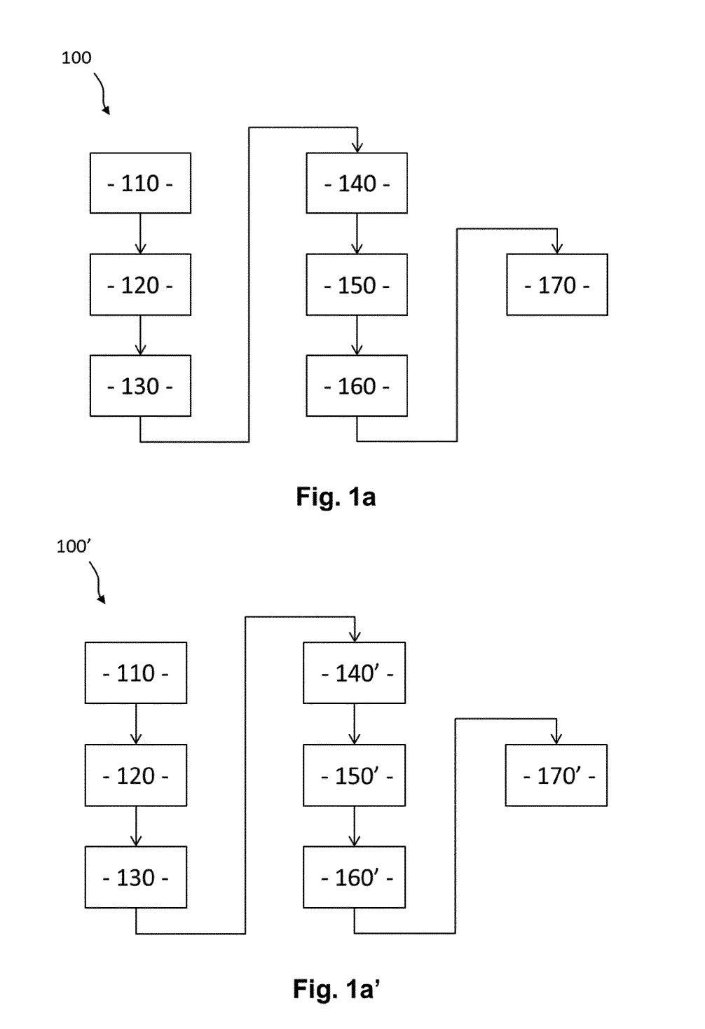

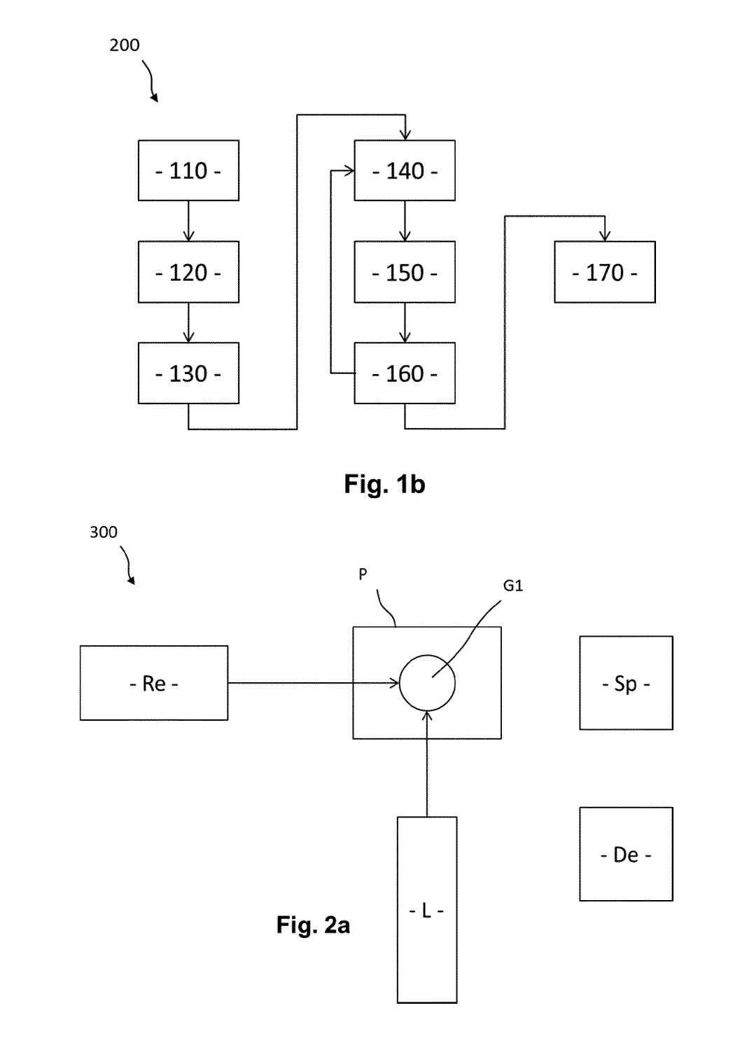

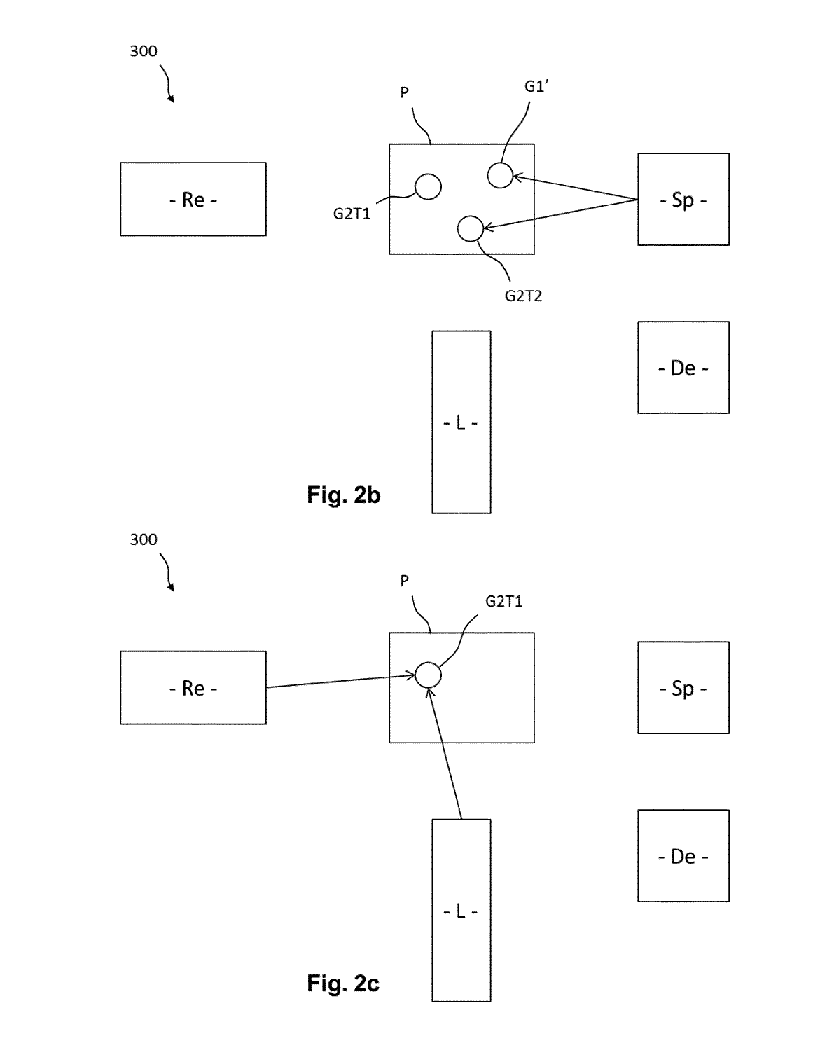

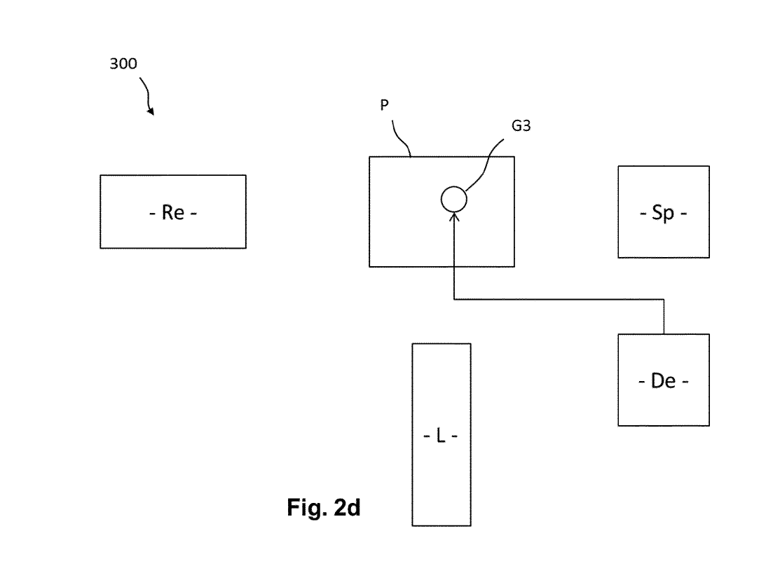

FIG. 1 a shows a diagram of the steps of a method 100 for characterising ions according to a first embodiment of the invention. FIGS. 2a, 2b, 2c and 2d show a use of a device 300 for characterising ions for the implementation of the method for characterising ions 20 according to the first embodiment of the invention. FIGS. 1a and 2a to 2d are described jointly.

FIGS. 1a and 2a show a step 110, according to which a plurality G1 of first-generation ions is trapped in an ion trap P. The plurality G1 of first-generation ions are in the form of a cloud of ions within the ion trap P.

The plurality G1 of first-generation ions is typically obtained using an electrospray ionisation (ESI) technique.

The ion trap P is preferentially a quadrupole trap, or Paul trap, which enables good localisation of a cloud of ions there within. A good localisation of the cloud of ions within the ion trap enables efficient interaction between a photo-fragmentation laser and the cloud of ions. Alternatively, the ion trap P may also be a Penning trap, or a multipolar linear trap.

The method 100 according to the first embodiment of the invention may advantageously comprise a step (not represented) according to which the plurality G1 of first-generation ions is selected so as to obtain a plurality G1 of first-generation ions of a first type. The step of selecting a first type of first-generation ions is for example carried out by a mass spectrometry technique. The step of selection by mass spectrometry advantageously makes it possible to obtain a plurality G1 of first-generation ions all having the same mass/charge ratio, noted m/z. The step of selection by mass spectrometry may for example take place before the plurality G1 of first-generation ions is introduced and trapped in the ion trap P. Alternatively, the step of selection by mass spectrometry may also take place after the plurality G1 of first-generation ions has been introduced and trapped in the ion trap P. In the latter case, the step of selection consists in conserving, in the ion trap P, first-generation ions having the desired m/z ratio by ejecting, out of the ion trap P, first-generation ions not having the desired m/z ratio. The step of selection of a first type of first-generation ions may also be carried out by an ion mobility spectrometry (IMS) technique.

FIGS. 1a and 2a show a step 120 according to which the plurality G1 of first-generation ions trapped in the ion trap P is cooled by means of a cooling module Re. The cooling module Re comprises:
a buffer gas source making it possible to introduce a buffer gas into the ion trap P, and
a cryostat.

The buffer gas is for example helium He. The buffer gas source may thus be a compressed helium cylinder. Alternatively, the buffer gas source may be a helium cylinder at ambient pressure which is used with a compressor.

FIGS. 1a, 2a and 2b show a step 130 according to which the plurality G1 of cooled first-generation ions is photo-fragmented by means of a photo-fragmentation laser L emitting at a first wavelength λ1, to obtain a plurality of second-generation ions. The first wavelength λ1 is selected as a function of the plurality G1 of first-generation ions to photo-fragment. The plurality of second-generation ions is different to the plurality of first-generation ions, and the plurality of second-generation ions is at least of one first type. At the end of the photo-fragmentation of the first-generation ions, the particular example represented in FIGS. 2a and 2b shows that the ion trap contains:
a first type G2T1 of second-generation ions,
a second type G2T2 of second-generation ions, and
residual first-generation ions G1′, which have not been photo-fragmented.
   Naturally, in another specific example (not illustrated), the ion trap P could contain third, fourth, . . . nth types of second-generation ions at the end of the photo-fragmentation of the first-generation ions. The ion trap P could also not contain any residual first-generation ion, or only contain a single type of second-generation ions.

FIGS. 1a, 2b and 2c show a step i), which is referenced 140 in FIG. 1a, according to which the first type of second-generation ions G2T1 is selected in the ion trap by ejecting, out of the ion trap, any residual first-generation ion and any second-generation ion of a type different to the first type. The step of selecting the first type of second-generation ions G2T1 is carried out using a mass spectrometer Sp. In the particular example illustrated in FIGS. 2b and 2c, the mass spectrometer Sp enables the ejection of residual first-generation ions G1′ and the second type of second-generation ions G2T2. Whatever the content of the ion trap P before the selection step 140, it is guaranteed that the ion trap P now only substantially contains the first type of second-generation ions G2T1 at the end of said selection step 140. “Substantially” is taken to mean the fact that a small residual quantity ε of ions to eject may subsist within the ion trap P after the selection step 140. The small residual quantity ε is sufficiently small so that its signal does not disturb the measurement. The small residual quantity ε is preferentially below 10% of all the ions to eject that were found in the ion trap P before the selection step 140.

The selection typically takes place by adding a radiofrequency voltage to an electrode of the ion trap P. This radiofrequency voltage is selected and adjusted to eject ions having a certain m/z ratio. In the particular example illustrated in FIGS. 2b and 2c, a first radiofrequency voltage may be applied to eject residual first-generation ions GI, then a second radiofrequency voltage may be applied to eject the second type of second-generation ions G2T2. Alternatively, the first and second radiofrequency voltages may be applied simultaneously.

FIGS. 1a and 2c show a step ii), which is referenced 150 in FIG. 1a, according to which the second-generation ions of the first type G2T1 selected and trapped in the ion trap are cooled, by means of the cooling module Re described previously.

FIGS. 1a, 2c and 2d show a step iii), which is referenced 160 in FIG. 1a, according to which the cooled second-generation ions of the first type G2T1 are photo-fragmented by means of the photo-fragmentation laser L emitting at a second wavelength λ2, to obtain a plurality of third-generation ions G3. The plurality of third-generation ions G3 is different to the plurality of second-generation ions, and the plurality of third-generation ions G3 is at least of one first type. The second wavelength λ2 is selected as a function of the second-generation ions of the first type G2T1 to photo-fragment. The second wavelength λ2 is typically different to the first wavelength λ1.

In the case that has just been described, the photo-fragmentation laser L emits at the first wavelength λ1 for the first photo-fragmentation of step 130, and emits at the second wavelength λ2 for the second photo-fragmentation of step 160. Alternatively, two photo-fragmentation lasers may be used: a first photo-fragmentation laser emitting at the first wavelength λ1 for the first photo-fragmentation of step 130, and a second photo-fragmentation laser emitting at the second wavelength λ2 for the second photo-fragmentation of step 160.

FIGS. 1a and 2d show a step 170 according to which the plurality of last-generation ions, in this case the plurality of third-generation ions G3, is detected by means of a detector De. The detection of the plurality of last-generation ions may for example be carried out by means of a channel photomultiplier (CPM), a multichannel plate (MCP) or a Daly detector. The spectroscopic signature of the first type G2T1 of second-generation ions is thereby obtained.

In the particular example that has just been described, a first type G2T1 of second-generation ions and a second type G2T2 of second-generation ions are obtained at the end of the photo-fragmentation of the first-generation ions G1. After having obtained the spectroscopic signature of the first type G2T1 of second-generation ions by carrying out a first cycle of steps 110 to 170, it is then possible to obtain the spectroscopic signature of the second type G2T2 of second-generation ions by carrying out a second cycle 100′, represented in FIG. 1a′, comprising:
the step 110, according to which the plurality G1 of first-generation ions is trapped in the ion trap P;
the step 120, according to which the plurality G1 of first-generation ions trapped in the ion trap P is cooled;
the step 130, according to which the plurality G1 of cooled first-generation ions is photo-fragmented to obtain, in the particular example considered, the first type G2T1 of second-generation ions and the second type G2T2 of second-generation ions;
a step 140′, according to which the second type G2T2 of second-generation ions is selected in the ion trap P by ejecting, out of the ion trap, any residual first-generation ions and any second-generation ion of a type different to the second type;
a step 150′, according to which the second-generation ions of the second type selected and trapped in the ion trap P are cooled;
a step 160′, according to which the cooled second-generation ions of the second type are photo-fragmented to obtain a second plurality of third-generation ions G3′;
a step 170′, according to which the second plurality of third-generation ions G3′ is detected.
   The spectroscopic signature of the second type G2T2 of second-generation ions is thereby obtained.

In the same way as described for step 140, the mass spectrometer Sp is used during step 140′. Whatever the content of the ion trap P before the selection step 140′, it is guaranteed that the ion trap P now only substantially contains the second type of second-generation ions G2T2 at the end of said selection step 140′. “Substantially” is taken to mean the fact that a small residual quantity ε of the ions to eject may subsist within the ion trap P after the selection step 140, as described previously for step 140.

In the same way as described for step 150, the cooling module Re is used during step 150′.

The photo-fragmentation laser L emitting at a third wavelength λ2′ is used during step 160′. The third wavelength λ2′ is selected as a function of the second-generation ions of the second type G2T2 to photo-fragment. The third wavelength λ2′ is typically different to the first wavelength λ1 and the second wavelength λ2.

In the case that has just been described, the photo-fragmentation laser L emits at the first wavelength λ1 for the first photo-fragmentation of step 130, emits at the second wavelength λ2 for the second photo-fragmentation of step 160 and emits at the third wavelength λ2′ for the third photo-fragmentation of step 160′. Alternatively, two photo-fragmentation lasers may be used: a first photo-fragmentation laser emitting at the first wavelength λ1 for the first photo-fragmentation of step 130, and a second photo-fragmentation laser emitting at the second wavelength λ2 for the second photo-fragmentation of step 160 and emitting at the third wavelength λ2′ for the third photo-fragmentation of step 160′. According to another alternative, three photo-fragmentation lasers may be used: a first photo-fragmentation laser emitting at the first wavelength λ1 for the first photo-fragmentation of step 130, a second photo-fragmentation laser emitting at the second wavelength λ2 for the second photo-fragmentation of step 160 and a third photo-fragmentation laser emitting at the third wavelength λ2′ for the third photo-fragmentation of step 160′.

In the same way as described for step 170, the detector De is used during step 170′.

Generally speaking, advantageously as many cycles may be carried out as different types of second-generation ions, so as to obtain the spectroscopic signature of each type of second-generation ion.

FIG. 1b shows a diagram of the steps of a method 200 for characterising ions according to a second embodiment of the invention. In the second embodiment of the invention, steps i), ii) and iii), which are respectively referenced 140, 150 and 160 in FIGS. 1a and 1b, are carried out sequentially N times, N being a natural integer greater than or equal to 2. The number of each generation is incremented by 1 each time said sequence is carried out.

An exemplary embodiment of the method 200 according to the second embodiment will now be described, according to which the number N of times the sequence of steps i), ii) and iii) is carried out is equal to 2.

The first chain of steps 110, 120, 130, 140, 150 and 160 of the method 200 according to the second embodiment is identical to the chain of steps 110, 120, 130, 140, 150 and 160 of the method 100 according to the first embodiment, which has been described previously. Indeed, for N=1, the method 200 according to the second embodiment is identical to the method 100 according to the first embodiment. At this stage, the sequence of steps i), ii) and iii) has thus been carried out once. For N=2, the sequence of steps i), ii) and iii) is next carried out a second time:
according to the second step i), the first type of third-generation ion is selected in the ion trap by ejecting, out of the ion trap, any residual second-generation ion and any third-generation ion of a type different to the first type;
according to the second step ii), the third-generation ions of the first type selected and trapped in the ion trap are cooled;
according to the second step iii), the cooled third-generation ions of the first type are photo-fragmented to obtain a plurality of fourth-generation ions, the plurality of fourth-generation ions being different to the plurality of third-generation ions, the plurality of fourth-generation ions being at least of one first type.

Finally, the method 200 according to the second embodiment of the invention comprises step 170, according to which the plurality of last-generation ions, in this case the plurality of fourth-generation ions, is detected. The spectroscopic signature of the first type of third-generation ions is thereby obtained. Generally speaking, the spectroscopic signature of each type of third-generation ion is advantageously determined.

1. A method for characterising ions comprising:
trapping a plurality of first-generation ions in an ion trap;
cooling the plurality of first-generation ions trapped in the ion trap;
photo-fragmenting the plurality of cooled first-generation ions to obtain a plurality of second-generation ions, the plurality of second-generation ions being different to the plurality of first-generation ions, the plurality of second-generation ions being at least of one first type;
selecting the first type of second-generation ions in the ion trap by ejecting, out of the ion trap, any residual first-generation ion and any second-generation ion of a type different to the first type;
cooling the second-generation ions of the first type selected and trapped in the ion trap;
photo-fragmenting the cooled second-generation ions of the first type to obtain a plurality of third-generation ions, the plurality of third-generation ions being different to the plurality of second-generation ions, the plurality of third-generation ions being at least of one first type;
detecting a plurality of last-generation ions.

2. The characterization method according to claim 1, further comprising selecting the plurality of first-generation ions by a mass spectrometry technique.

3. The characterization method according to claim 1, wherein the plurality of last-generation ions is detected by a mass spectrometry technique.

4. The characterization method according to claim 1, wherein the selecting of the first type of second-generation ions, the cooling of the second-generation ions of the first type and the photo-fragmenting of the cooled second-generation ions of the first type form a sequence that is carried out N times, N being a natural integer greater than or equal to 2, the number of each generation being incremented by 1 each time said sequence is carried out.

5. A device for characterising ions for the implementation of the method for characterising ions according to claim 1, the device comprising:
an ion trap for trapping a plurality of ions;
a module for cooling the plurality of ions, comprising a buffer gas source and a cryostat;
a photo-fragmentation laser;
a mass spectrometer for selecting a type of ions of interest;
a detector for detecting a plurality of photo-fragments.
